(19) United States
(12) Patent Application Publication
(10) Pub. No.: US 2007/0183978 A1
(43) Pub. Date: Aug. 9, 2007

(54) METHODS AND COMPOSITIONS FOR DIAGNOSIS AND IMMUNOTHERAPY OF POLLEN ALLERGY

(75) Inventors: Daphne Preuss, Chicago, IL (US); Ravishankar Palanivelu, Tucson, AZ (US); Katinka A. Vigh, Chicago, IL (US); Matthew A. Cummings, Chicago, IL (US); Mohamed E. Bashir, Oak Lawn, IL (US)

Correspondence Address: BARNES & THORNBURG LLP, P.O. BOX 2786, CHICAGO, IL, 60690-2786, US

(73) Assignee: THE UNIVERSITY OF CHICAGO, Chicago, IL (US)

(21) Appl. No.: 11/530,627

(22) Filed: Sep. 11, 2006

Related U.S. Application Data
(60) Provisional application No. 60/715,650, filed on Sep. 9, 2005.

(51) Int. Cl. A61K 49/00; C07K 14/415

(52) U.S. Cl. 424/9.81; 530/370

(57) ABSTRACT

A diagnostic pollen array includes allergens extracted from pollen coat material and pollen cytoplasm. Diagnostic pollen arrays are useful to diagnose allergy in individuals, identify novel allergens, identify genetic loci responsible for allergy in hosts, and to develop personalized treatment plans for allergy.

This application claims priority to U.S. Ser. No. 60/715,650, filed Sep. 9, 2005.

### BACKGROUND

Current allergy diagnostics, such as the skin prick test (SPT) or radioallergosorbent test (RAST), typically rely on proteins soluble in aqueous buffers that are extracted from washed and lysed pollen grains. Similar extraction methods are often used to provide reagents for immunotherapy, a treatment for long-term allergy relief.

Knowledge of animal host genes that contribute to allergy susceptibility could facilitate the development of new, more effective allergy treatments for patients. An understanding of pollen components or pollen materials can provide important clues to their role in triggering allergy and asthma. The walls of pollen grains are composed of an unusual polymer coated with a lipid-rich layer that contains a small set of proteins. When this layer or surface contacts female flower cells, it rapidly diffuses; enzymes contained within this surface layer have the potential to alter pollen contents, as well as the composition of cell surfaces they contact. Pollen grains can absorb water from cells they contact and begin releasing intracellular enzymes that enable the extension of a pollen tube that carries sperm.

### SUMMARY

Pollen allergen arrays disclosed herein accurately assess patients' sensitivity to a wide range of pollen allergens, including organic extracts from unwashed pollen. Non-pollen allergens, e.g., mite, dust, dander and the like can also be included in the diagnostic microarrays disclosed herein. Patient sensitivity to pollen allergen data, collected by screening individuals using a pollen array, define a specific and semi-quantitative pollen sensitization phenotype for identifying pollen susceptibility genes, either with linkage mapping or association studies.

One method for identifying genes that contribute to allergy is to correlate patient responses with genomic DNA markers. The pollen array described herein can be used to analyze sera from any group of patients, by measuring the presence of antibodies or any suitable immunological marker, to specific pollen components. When combined with genetic information, this phenotype data can be used to map or identify allergy susceptibility genes.

Compositions and methods relate extraction of pollen components and use of such extracted pollen components to diagnose allergen sensitization, to identify novel gene products in pollen, to identify allergy-specific genetic markers in hosts, and to develop allergy treatments. A collection of extracted pollen components including organic solvent extraction, for example, in an array format disclosed herein, diagnose allergen sensitization in one or more individuals.

Pollen extracts routinely used for allergy diagnostics and therapy do not contain components extracted with organic solvents from unwashed pollen. Pollen arrays described herein include pollen components extracted from multiple allergenic species and are prepared by extraction of pollen components from unwashed pollens using an organic solvent. Pollen arrays described and disclosed herein include pollen allergens that are absent from commercially available traditional pollen extracts. Pollen arrays described and disclosed herein include pollen allergens from organic extracts and aqueous extracts of pollen components.

A pollen array includes a pollen surface allergen. The pollen array is a diagnostic pollen array. The allergen is reactive to an IgE antibody. The allergen is present in a pollen extract. The pollen extract includes a pollen cell surface extract. The pollen extract also includes a pollen cytoplasmic extract. A pollen cell surface extract is obtained using an organic solvent. A pollen cytoplasmic extract is obtained using an aqueous solvent.

A pollen allergen present in a pollen array includes a recombinant pollen allergen. The allergen is substantially pure or highly pure. The pollen allergen in the array includes a peptide and/or a multimeric pollen allergen. The pollen allergen in the array is present in a concentration of about 0.05-1.0 μg/L. The pollen allergen in the array is present in an amount sufficient to detect an allergy response in a patient sample. A pollen array includes a plurality of pollen allergens spotted at a density of about 100 spots per square inch to about 100,000 spots per square inch or at a density of about 1000 spots per square inch to about 10,000 spots per square inch.

A pollen array includes a pollen allergen selected from cytoplasmic, cell-wall bound, and membrane bound allergens. A pollen allergen is selected fro example from a group of allergenic plant species Wal (Walnut), Ber (Bermuda grass), Scy (Sycamore), Orc (Orchard grass), Pec (Pecan), Tim (Timothy grass), Olv (Olive), Mug (Mugwort), WOk (White Oak), Rag (Ragweed), Ald (Alder), Eld (Box Elder), Cot (Cottonwood), Mul (Mulberry), Jhn (Johnson grass), Elm, Ash, Ced (Cedar), Blu (Bluegrass), Bir (Birch), Rye, and ROk (Red Oak). Any plant species with pollen is suitable for use in the pollen arrays described herein. A pollen array, in an embodiment, includes at least one cytoplasmic allergen and an antibody standard.

A method of making a pollen array includes the steps of:
(a) obtaining pollen from an allergenic plant species;
(b) preparing a pollen cell surface extract from the pollen;
(c) obtaining at least one pollen allergen from the pollen extract; and
(d) applying the pollen allergen to a solid support in an orderly fashion in the array.

A pollen used in making a pollen array is untreated and unwashed. The pollen is non-defatted.

A pollen cell surface extract is prepared using an organic solvent selected from cyclohexane, hexane, diethylether, formamide, dimethylformamide, dimethyl sulfoxide, acetone, ethyleneglycol monomethyl ether, toluene, benzene, hydrocarbon solvents and halogenated solvents. Any suitable organic solvent is useful in preparing a cell surface extract described herein.

A solid support for an array includes for example, glass, epoxy-coated glass, plastic, nylon and nitrocellulose membrane.

A method of preparing a pollen surface allergen extract includes the steps of:
(a) obtaining non-defatted unwashed pollen from an allergenic plant species;
(b) exposing the non-defatted pollen to an organic solvent to separate the surface pollen; and
(c) obtaining the surface pollen allergen extract.

A pollen surface extract is substantially free of cytoplasmic components. A pollen extract is substantially completely free of cytoplasmic components.

A method of preparing a pollen extract including pollen surface allergens and pollen cytoplasmic allergens includes the steps of:
(a) obtaining pollen from an allergenic plant species;
(b) washing the pollen with an organic solvent to separate the surface pollen allergens;
(c) lysing the washed pollen in an aqueous solution to obtain the cytoplasmic allergens;
(d) obtaining the pollen extract including the pollen surface allergens and pollen cytoplasmic allergens.

A method to measure antibody levels to pollen allergens in an individual includes the steps of:
(a) providing a pollen array including a pollen surface allergen;
(b) applying a serum sample from the individual to the pollen array; and
(c) measuring antibody levels to the allergen by quantifying allergen-antibody reactions on the array.

A method of measuring sensitivity to pollen allergens in an individual includes the steps of:
(a) providing a pollen array including at least one pollen surface allergen;
(b) applying a serum sample from the individual to the pollen array; and
(c) analyzing the sensitivity to pollen allergen by measuring antibody levels to the allergen.

A method of identifying a genetic locus that contributes to allergy includes the steps of:
(a) diagnosing allergen sensitization in a group of individuals using a pollen array;
(b) correlating results of the diagnosing to at least one genetic marker linked to a locus; and
(c) identifying the genetic locus that contributes to allergy.

A method to develop a pollen-specific allergy treatment in an individual includes the steps of:
(a) providing a pollen array including at least one pollen surface allergen;
(b) identifying a pollen allergen or a group of pollen allergens that contribute to allergy in the individual; and
(c) developing a therapy to treat allergy to the pollen allergen in the individual.

A group of pollen allergens are selected based on their reactivity to the individuals IgE antibodies in developing a pollen-specific allergy treatment in an individual. A pollen-specific allergy treatment is personalized to the individual.

A method to treat an individual allergic to one or more specific pollen allergens includes the steps of:
(a) providing the pollen array including at least one pollen allergen to develop an allergy profile of the individual;
(b) administering one or more pollen allergens identified in (a) at a dosage that is sufficient to elicit a desensitization response in the individual; and
(c) treating the individual by administering progressively higher doses of allergen and monitoring the hyposensitization response in the individual to the pollen allergen using the pollen array.

An isolated allergenic pollen peptide from Bermuda grass includes an amino acid sequence WVIENGGITTLADYPYR. The allergenic pollen peptide is synthetic and is substantially free of other allergenic peptides.

An immunologically active composition includes an allergenic peptide of amino acid sequence WVIENGGITTLADYPYR. The immunologically active composition of includes the allergenic peptide in an amount that is effective to induce hyposensitization in an individual. The allergenic pollen peptide includes an amino acid sequence that is more than 95% identical to WVIENGGITTLADYPYR. The allergenic pollen peptide includes an amino acid sequence that is more than 90% identical to WVIENGGITTLADYPYR. or more than 85% identical to WVIENGGITTLADYPYR.

A pollen allergen is extracted using a solvent. The solvent includes organic and inorganic solvents. The solvent is selected from a group that includes polar, non-polar, protic, and aprotic solvents. An organic solvent is selected from a group of solvents that includes for example, cyclohexane, hexane, diethylether, formamide, dimethylformamide, dimethyl sulfoxide, acetone, ethanol, methanol, ethyleneglycol monomethyl ether, toluene, benzene, any suitable hydrocarbon solvents or halogenated solvents.

A pollen allergen may also be extracted using any suitable method that captures pollen coat, cell wall-bound, membrane-bound, intracellular, and extracellular pollen material. Pollen extract materials can also be obtained or extracted using reagents from commercial suppliers, using their proprietary or FDA-approved methods so long as the reagents and the methods enable isolation of all the pollen components and fractions disclosed herein.

### BRIEF DESCRIPTION OF THE DRAWINGS

The drawings are provided to illustrate some of the embodiments of the disclosure. It is envisioned that alternate configurations of the embodiments of the present disclosure are within the scope of the disclosure.

FIG. 1 represents a low-density pollen array. Components extracted from washed pollen were blotted in duplicate spots onto nitrocellulose and probed with sera from non-allergic (1) or allergic (2, 3) individuals (Left panel). Sensitivity to four pollen species was tested: Tim (Timothy grass), Rag (Ragweed), WOk (White Oak) and Olv (Olive). Dark spots indicate the individual sera contain IgE antibodies that react with the pollen material. Components extracted from washed (W) or unwashed (UW) pollen were blotted in duplicate spots onto nitrocellulose and probed with antisera pooled from 500 individuals (Catalog HMSRM, Bioreclamation Inc., Hicksville, N.Y.). (Right panel) 22 pollen species were tested, including Wal (Walnut), Ber (Bermuda grass), Scy (Sycamore), Orc (Orchard grass), Pec (Pecan), Tim (Timothy grass), Olv (Olive), Mug (Mugwort), WOk (White Oak), Rag (Ragweed), Ald (Alder), Eld (Box Elder), Cot (Cottonwood), Mul (Mulberry), Jhn (Johnson grass), Elm, Ash, Ced (Cedar), Blu (Bluegrass), Bir (Birch), Rye, and ROk (Red Oak). Controls in which the human sera or the secondary anti-IgE antibody were spotted directly onto the nitrocellulose were also included.

FIG. 2 is a schematic representation of a high-density pollen array and shows the layout of the diagnostic pollen array. Allergens are spotted at a concentration of about 0.3 μg/mL. Samples under column “a” refers to commercial pollen extracts; “b” refers to cytoplasmic pollen extracts obtained using the methods described herein; “c” refers to surface pollen extracts obtained using the methods described herein; “d” refers to commercial non-pollen extracts; shaded cells refer to commercial recombinant allergens; “HSA” serves as a negative control; and antibody standards are identified as IgE, IgG, and IgA. The amount of standards spotted is indicated in picograms.

FIG. 3 shows images of scanned allergen arrays showing IgE reactivity to 80 different allergens in triplicate. 10 μL of serum from two different individuals diluted to 20% was added to the allergen arrays and IgE binding was visualized using a fluorescently labeled anti-IgE secondary antibody. Organization pattern of the array shown is based upon FIG. 2.

FIG. 4 shows comparison of IgE standard curves that demonstrate reliability and accuracy of data analysis on diagnostic pollen arrays. The curve represents the average of IgE standards on 96 arrays on 8 slides. Error bars represent standard deviation from the mean.

FIG. 5 is graph showing pollen reactivity profiles for 3 individuals. Reactivity to cytoplasmic and surface components from 5 pollens is shown as different hatchings and shadings. Bound IgE is extrapolated from the median fluorescent intensity of the IgE standard curves.

FIG. 6 shows gel images of allergens isolated from the surface and cytoplasmic fractions of Bermuda grass pollen proteins. Stained image (A) and western blot (B) show distinct bands. The western blot shows that three proteins from the pollen surface and one protein from the cytoplasm are recognized by the human IgE in a pooled sera screening. The numbers 1, 2, and 3 represent cell surface-specific endoxylanase (1), allergen Cyn d 1 (2) and cysteine protease (3) respectively. The IgE binding proteins were identified via peptide fingerprinting on a matrix assisted laser desorption/ionization-time of flight (MALDI-TOF) instrument and direct sequencing.

FIG. 7 shows total IgE reactivity to different pollen extracts. The total IgE reactivity added across 21 allergens and 24 individuals is plotted. Paired student T test indicates that reactivity to cytoplasmic extracts obtained using the methods described herein is significantly higher than to commercial extracts (p=5.2×10⁻⁴).

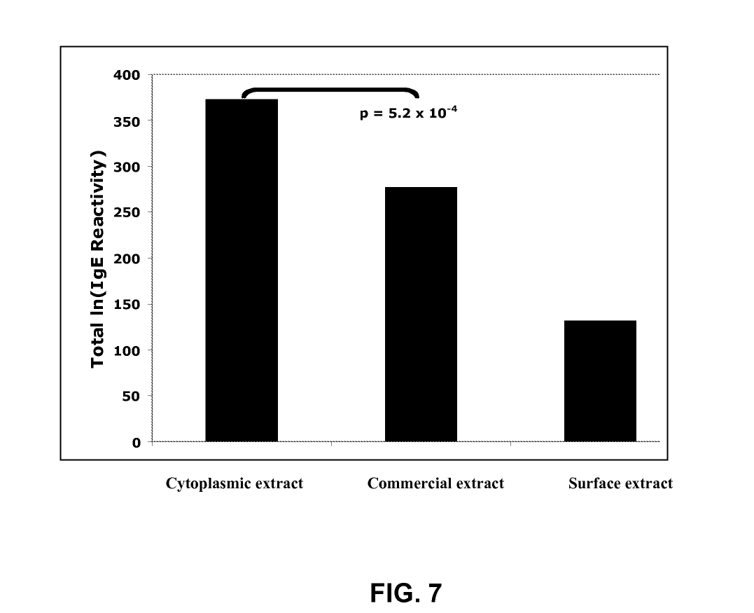

FIG. 8 shows hierarchical clustering analysis performed using Heatmap on IgE reactivity data from 24 individuals (A). 5 recombinant allergens (Derm-p-mite, Rag-ragweed, Tim-timothy grass, Bir-birch, Alt-alt-mold) or B. cytoplasmic extracts from 21 pollens prepared as described herein. All plants indicated are angiosperms except cedar. Within the angiosperms, Mul (mulberry), Olive, Alder, R-Oak (red oak), Syc (sycamore), Elm, W-oak (white oak), Rag (ragweed), Cot (cottonwood), Elder, Ash, Pecan, Bir (Birch), and Mug (mugwort) are dicots, and Orch (orchard grass), Tim (timothy grass), John johnson grass), Blue (bluegrass), Rye (ryegrass), and Ber (bermuda grass) are monocots.

### DETAILED DESCRIPTION

Methods and compositions relating to diagnostic pollen arrays involve developing arrays that include allergens from unwashed pollen, the pollen surface or pollen coat. The pollen arrays disclosed herein that contain both intracellular, extracellular and pollen coat-specific allergens, are useful to diagnose allergen sensitization with an increased level of accuracy and specificity, identify associated or linked host genetic markers and develop treatment plans that are targeted towards particular allergy responses.

Diagnostic pollen arrays disclosed herein are constructed by selecting allergenic pollen species, collecting and extracting pollen components or pollen extracts from the pollen and constructing variable density pollen arrays.

Pollen extracts disclosed herein are used for several purposes that include (i) providing a non-invasive diagnostic tool to measure a patient's sensitivity to pollen allergens; (ii) collecting patient response data that facilitate identification of genes that contribute to allergy; (iii) identifying and purifying novel allergens; (iv) studying B cell pathways; (v) measuring patient IgE, IgA, and IgG antibodies; and (vi) monitoring antibody responses during immunotherapy.

The pollen material or component extraction and isolation methods disclosed herein improve the accuracy of allergy diagnosis and the effectiveness of allergy therapy. The extraction and isolation methods disclosed herein maximize the capture of pollen allergens that otherwise would be lost in a traditional isolation and extraction procedure. For example, the capture of pollen surface allergens that include pollen coat proteins, or pollen wall material is enhanced. The extraction and isolation methods disclosed herein are designed to capture intracellular, extracellular, cell wall bound, and membrane bound pollen components from washed and unwashed pollens that may contribute to allergy.

A diagnostic pollen array is designed, for example, by applying or affixing pollen components to a solid support. A pollen array can include any form of support, such as for example, glass, plastic, nylon or nitrocellulose membrane or any similar support. An array as used in herein encompasses any ordered arrangement of pollen allergens or combinations of allergens. The ordered arrangement can include a low density array that may contain at least one pollen allergen, or a high density array that contains a plurality of pollen allergens. An array may also contain allergens in duplicates or multiple replicates, allergens that are derived from sources other than pollen, and may also contain allergenic and non-allergenic controls. Components may be spotted on a support or applied or affixed in any manner dependant on the type of support.

Allergens as used herein include proteins, peptides, carbohydrates, a combination thereof, or any biochemical factor capable of triggering a measurable allergic response. Allergens may also include both allergens purified to homogeneity, as well as allergens in crude extracts that contain mixtures of allergenic and non-allergenic components. These biochemical factors or allergens, or fragments thereof, can be intracellular, extracellular, membrane bound, or cell wall bound. Few pollen allergens have been directly tested for their ability to cause allergy and the possibility exists that they could merely cross react with commonly-produced IgEs or play secondary, but not causative roles. Molecules (peptides, proteins, carbohydrates or a combination or a fragment thereof) identified following the methods disclosed herein to cause allergenic responses are tested for pulmonary and cellular responses. A mouse model is a suitable system considered representative of human allergy. Promising molecules are tested in vivo by examining the ability of the allergens to provoke airway inflammation.

In an illustrative example, a pollen array contains both intracellular components extracted from washed pollen, as well as components extracted from unwashed pollen. Preparations from several individual pollen species, spotted in either a high or low-density format, are included in the arrays. Sera from allergic or non-allergic patients are applied to the array, and the presence of antibodies is measured, providing an assessment of the patient's sensitivity to specific pollen species.

Allergenic pollen species are selected through available knowledge in the literature. For example, a wide range of allergenic pollen species are known, including grasses, weeds, and trees. An exemplary list of pollen allergens is given in Table 1, disclosed herein. The OPALS™ database (Ogren, 2000) indicates the extent of pollen allergenicity from a wide range of species, with a score of 10 being the most allergenic. Components from pollen described in this database, as well as from other pollen species, are extracted as disclosed herein.

“Array or microarray” relates to molecules generally connected to a matrix or support (substrate) in a specific arrangement relative to each other. Any substrate including for example, glass, processed glass, coated glass, plastic, fiber, polymer, gel, and membranes are suitable for use in an array.

“Allergen” means any substance that induces an allergy including proteins, peptides, peptide fragments, recombinant peptides, synthetic peptides or a combination thereof. These proteins and peptides can be either cytoplasmic, cell-wall bound, and membrane bound allergens. “Allergen” can also include modified proteins or peptides, where the modifications include lipids, carbohydrates or other alterations.

“Allergy” means an abnormal reaction of the body to a previously encountered allergen introduced by inhalation, ingestion, injection, or skin contact.

“Allergenic” means capable of eliciting an allergy.

“Commercially available extract” refers to mixtures of biological materials that are sold by commercial suppliers.

“Component” as used herein means an element forming part of a whole and “material” as used herein means a constituent of an element of a substance.

“Cytoplasmic extract” refers to mixtures of cytoplasmic allergens.

Cytoplasmic allergen refers to an allergen that is either minimally or substantially present in the cytoplasmic compartment of a pollen, or is a recombinant form of a protein that was initially identified in the cytoplasmic compartment of a pollen.

“Diagnostic pollen array”—a pollen array used for monitoring the presence of antibodies that react with allergens. This can be used to diagnose the status of an individual or groups of individuals, for diagnosis or therapeutic purposes, or for research activities.

“Highly pure” refers to a purity of about 80% or greater.

“Multimeric pollen allergen” refers to either homopolymeric and heteropolymeric forms of allergens that collectively present an allergenic epitope or epitopes. For example, one or more pollen allergens present on the cell surface, together, present an epitope or epitopes to trigger allergy. “Multimeric pollen allergen” also includes modified peptides (decorated for example with carbohydrates, lipids, or other modifications).

“Non-defatted” pollen refers to pollen that has not been treated to remove some or all of the lipophilic pollen components.

“Pollen surface extract” refers to mixtures of pollen surface allergens.

“Pollen array”—an assembly on a solid support of pollen components, comprised of allergens.

Peptides that are highly similar to the disclosed peptides are within the scope of the disclosure. For example, a peptide that is more than 95% identical, or 90% identical, or 85% identical is within the scope of the disclosure. These peptides include natural variations, artificial substitutions, deletions, insertions, mutations, and functional equivalents.

“Pollen surface allergen” refers to an allergen that is either minimally or substantially exposed to the exterior and present in the pollen surface or is a recombinant form of a protein that was initially identified in the pollen surface.

“Substantially pure” refers to a pollen surface allergen fraction that includes more than 90% of pollen surface components including proteinaceous pollen allergens.

“Substantially free” refers to a pollen surface allergen fraction that includes less than 10% of pollen cytoplasmic components.

“Substantially completely free” refers to a pollen surface allergen fraction that includes negligible amount of pollen cytoplasmic components (not easily detected, e.g., in a western blot).

“Untreated” pollen refers to pollen that has not been treated with any agent.

“Unwashed” pollen refers to pollen that has not been washed with any liquid reagent to remove pollen components.

### EXAMPLES

The following examples are illustrative and do not limit the scope of the various methods and compositions disclosed herein.

### Example 1

Extraction of pollen coat material. Extraction of pollen coat components is performed following the methods disclosed herein. To remove hydrophobic components, including those on the pollen surface, unwashed pollen was extracted with cyclohexane or any suitable organic solvent that is capable of extracting pollen coat components. Insoluble solid material is removed by centrifugation or filtration, and components dissolved into the organic phase are collected and concentrated by evaporation of the organic solvent under air or nitrogen. Residual components are precipitated with trichloroacetic acid (TCA), ammonium sulfate, acetone or other suitable reagents. To obtain components from the pollen cytoplasm, pollen washed with cyclohexane or other organic solvents such as dimethylformamide, carbon tetrachloride, or a combination thereof, is lysed in an aqueous buffer, using a mortar and pestle. Solid (insoluble) material is removed by centrifugation or filtration, and the soluble components are used directly, or are precipitated with trichloroacetic acid (TCA), acetone, ammonium sulfate, isopropanol or other suitable precipitating reagents. Precipitated components are dissolved in solutions containing potassium or sodium salts, buffered at a pH between 6 and 8, and containing about 5% or less of a non-ionic or an ionic detergent. Non-aqueous inorganic solvents like liquid ammonia and liquid sulfur dioxide are also suitable.

### Example 2

Pollen fractionation. Each pollen sample may initially be divided into three fractions: (a) extracellular components that are washed from the pollen grain with organic solvents as in Example 1 and as described in the Materials and Methods section; (b) components from lysed pollen grains that dissolve into aqueous buffers as described in Example 1 and in the Materials and Methods section; and (c) insoluble components, including the pollen wall. For the extracellular fraction, proteins are separated from lipids based on their differential solubility in isopropanol and chloroform, Intracellular fractions are used directly or are extracted with trichloroacetic acid, and/or polyvinylpolypyrrolidone and 2-mercaptoethanol and washed with cold acetone.

### Example 3

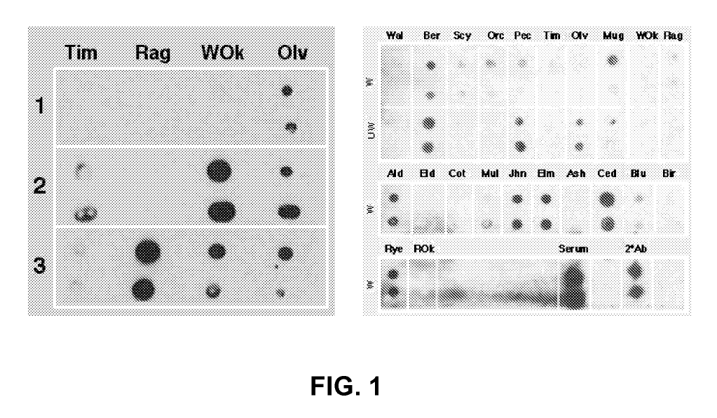

Construction of pollen arrays. For a low-density pollen array (see Materials and Methods), approximately 1-10 μl of suspended pollen components, extracted as described in Example 1 or Example 2, are spotted onto nitrocellulose or any suitable membrane to form a low-density pollen array. Membranes can be blocked with proteins such as bovine serum albumin or non-fat milk and incubated with individual or pooled human sera following standard procedures. Bound antibodies are detected with labeled anti-human secondary antibodies, such as anti-IgE, anti-IgG, or anti-IgA. In the example illustrated in FIG. 1, bound human antibodies were detected with a horseradish peroxidase (HRP) conjugated anti-human IgE secondary antibody and visualized with a luminescent substrate. The secondary antibody can be labeled with any detectable label. This experiment demonstrated distinct differences among patients in their IgE reactivity to pollen components, and sera pooled from 500 individuals demonstrated evidence of stronger sensitivity to components extracted from unwashed pollen, as compared to extracts from washed pollen.

For a high-density array (see Materials and Methods), pollen components can also be arranged on a support generally referred to as a microarray. Pollen components isolated from a variety of plant species can be arranged in duplicates or triplicates or in any other suitable format in a support. Generally, the support may be solid and includes glass, plastic, or any other suitable material. Spotting or affixing extracted pollen components can be performed using techniques known to a skilled artisan.

### Example 4

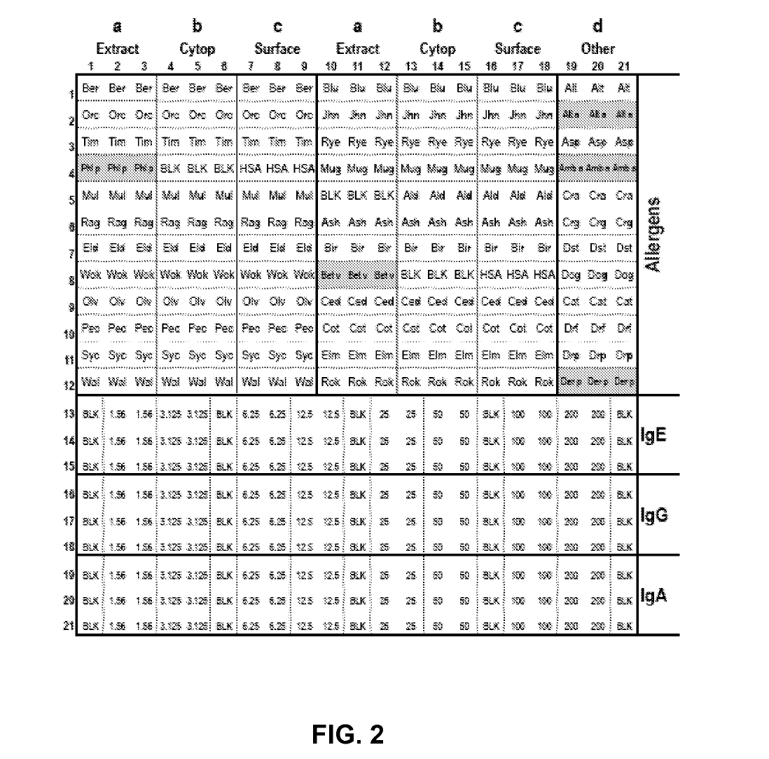

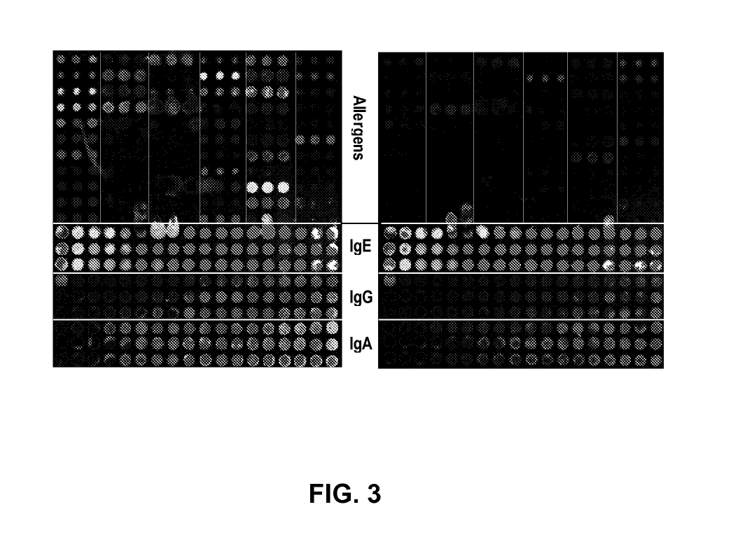

High density allergen arrays. A high-throughput high density protein microarray based assay to quantitatively measure allergen sensitization was developed. In the illustrated example shown in FIGS. 2 and 3, the array density was about 441 allergen spots (including controls) per about 6 square millimeters. Pollen surface and cytoplasmic materials isolated from 22 highly allergenic plant species have been spotted in triplicates onto glass slides at a concentration of 0.3-1.0 μg/μL (ArrayIt™, Sunnydale, Calif.). Depending on the sensitivity of the solvents, labeling agents, signal scanner, signal analysis, and noise reduction, the concentration of the pollen materials or other allergen or non-allergen spotted on the array can be lower or higher than the ranges disclosed herein. For example, the concentration of pollen allergens can further range from about 10 nanograms to about 100 nanograms/μl or from about 1.0 μg-10.0 μg/μl. Arrays have from about 100 spots to about 100,000 spots per square inch. Arrays also have from about 1000 spots to about 10,000 spots per square inch. Arrays can also have from about 1000 spots to about 20,000 spots per square inch. Commercially available extracts of the same pollens as well as 9 non-pollen and 5 recombinant allergens have also been included in the high density array. Unique reactivity in both the fractions prepared in the inventors' labs and the commercially available extracts has been observed. In addition to the allergens, 3 immunoglobulin standards were also spotted allowing for standardization of reactivity across different arrays. These are purified IgE, IgG, or IgA proteins—the primary antibody that the secondary antibody binds to. Quality and reproducibility of the allergen arrays disclosed herein are comparable or better than previous methods using recombinant proteins (FIG. 3).

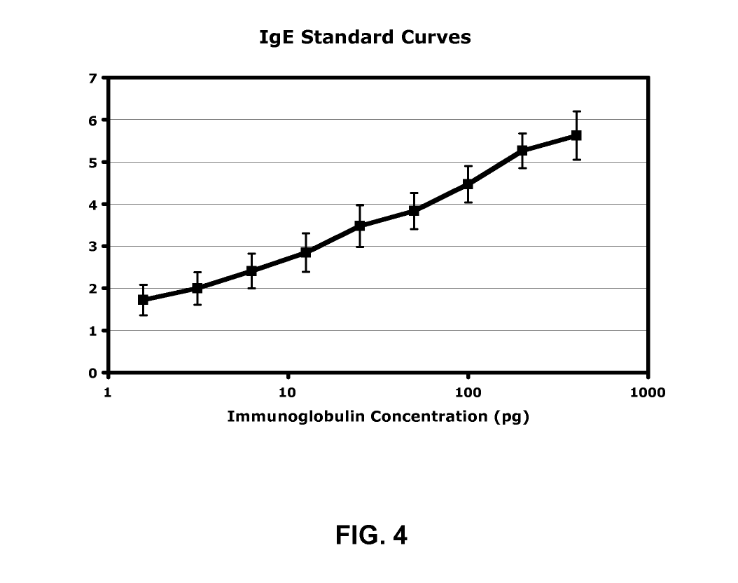

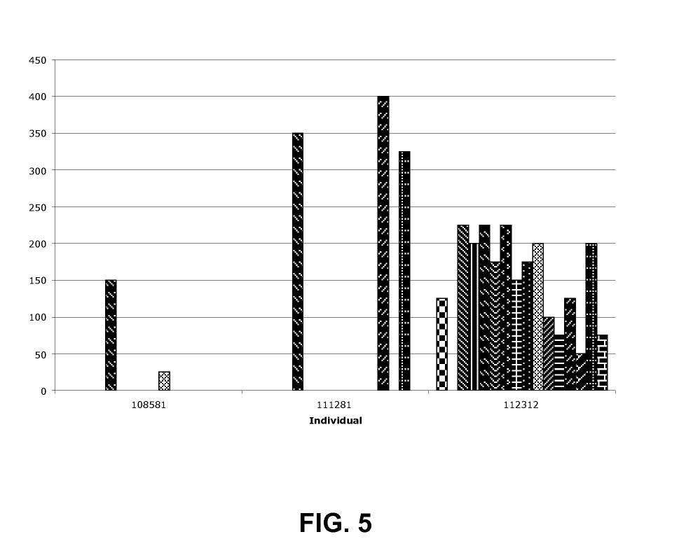

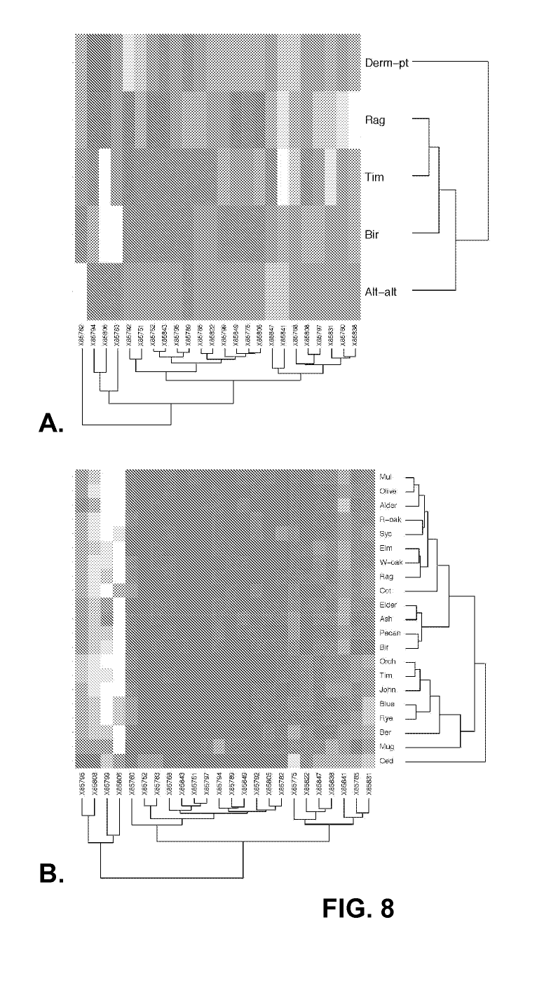

In the illustrated example on FIG. 3, a sample size of about 10-20 μl of human sera, diluted as needed, is sufficient to allow for the survey of reactivity to a wide range of pollen species, including all pollen components, and several non-pollen allergens. In this example, at least 80 different allergens can be tested at once. Allergen sensitization is detected with fluorescently labeled secondary antibody (anti human IgE, IgG1, IgG2, and IgA). Screening individual sera revealed distinct allergen sensitization profiles among individuals (FIGS. 5 and 8). In addition, cytoplasmic fractions extracted using the methods disclosed herein contained more allergenic material than was found in commercially available extracts (FIG. 10). FIG. 8 shows one embodiment of a layout of a diagnostic pollen array containing commercially available pollen extracts, cytoplasmic and surface pollen extracts prepared according to the methods disclosed herein, commercially available non-pollen extracts, negative controls and standard IgA, IgE, and IgG antibodies. The amount of the spotted standard antibodies is shown in picograms in FIG. 2. FIG. 4 shows comparison of IgE standard curves that demonstrate reliability and accuracy of data analysis on diagnostic pollen arrays. Quality and reproducibility of the diagnostic pollen allergen array is comparable or better than previous methods using recombinant allergens. FIG. 5 shows pollen reactivity profiles of three individuals to various pollen species (both cytoplasmic and surface fractions). The sera from these individuals were screened against the diagnostic pollen array disclosed herein. The individuals show specific and distinct response to pollen extracts from various pollen species.

A diagnostic pollen array described herein can have any number of pollen allergens. An exemplary list of pollen allergens from Greer's commercial catalog is provided in Table 1

The allergen arrays disclosed herein are an effective way of assessing the allergen sensitization phenotype of individuals and aid in mapping allergy susceptibility genes. Screening, for example, a highly inbred isolate is useful in identifying the genetic links to allergy. Comparable numbers of an urban, more ethnically diverse population, such as Chicagoans, can also be screened using the allergen arrays disclosed herein. Using these quantitative and multidimensional allergen sensitization phenotype phenotype-genotype correlations to identify genes that predispose people to allergy are performed.

TABLE 1
Exemplary list of allergenic plant species

GRASSES:
Bahia Paspalum notatum
Bermuda Cynodon dactylon
Blue, Canada Poa compressa
Brome, Smooth Bromus inermis
Canary Phalaris arundinacea
Corn Zea mays
Couch/Quack Elytrigia repens (Agropyron repens)
Johnson Sorghum halepense
Kentucky Blue Poa pratensis
Meadow Fescue Festuca pratensis (elatior)
Oat, Cultivated Avena sativa
Orchard Dactylis glomerata
Red Top Agrostis gigantea (alba)
Rye, Cultivated Secale cereale
Rye, Giant Wild Leymus (Elymus) condensatus
Rye, Italian Lolium perenne ssp. multiflorum
Rye, Perennial Lolium perenne
Sweet Vernal Anthoxanthum odoratum
Timothy Phleum pretense
Velvet Holcus lanatus
Wheat, Cultivated Triticum aestivum
Wheatgrass, Western Elymus (Agropyron) smithii
FLOWERS:
Daisy, Ox-Eye Chrysanthemum leucanthemum
Dandelion Taraxacum officinale
Sunflower Helianthus annuus
CULTIVATED PLANTS:
Alfalfa Medicago sativa
Castor Bean Ricinus communis
Clover, Red Trifolium pratense
Mustard Brassica spp.
Sugar Beet Beta vulgaris
WEEDS:
Baccharis Baccharis halimifolia
Allscale Atriplex polycarpa
Baccharis Baccharis sarothroides
Burrobrush Hymenoclea salsola
Careless Weed Amaranthus hybridus
Cocklebur Xanthium strumarium (commune)
Dock, Yellow Rumex crispus
Dog Fennel Eupatorium capillifolium
Goldenrod Solidago spp.
Hemp, Western Water Amaranthus tuberculatus (Acnida tamariscina)
Iodine Bush Allenrolfea occidentalis
Jerusalem Oak Chenopodium botrys
Kochia/Firebush Kochia scoparia
Lambs Quarter Chenopodium album
Marsh Elder, Burweed Iva xanthifolia
Marsh Elder, Narrowleaf Iva angustifolia
Marsh Elder, Rough Iva annua (ciliate)
Mexican Tea Chenopodium ambrosioides
Mugwort, Common Artemisia vulgaris
Mugwort, Darkleaved Artemisia ludoviciana
Nettle Urtica dioica
Palmer's Amaranth Amaranthus palmeri
Pigweed, Redroot/Rough Amaranthus retroflexus
Pigweed, Spiny Amaranthus spinosus
Plantain, English Plantago lanceolata
Poverty Weed Iva axillaris
Quailbrush Atriplex lentiformis
Rabbit Bush Ambrosia deltoidea
Ragweed, Desert Ambrosia dumosa
Ragweed, False Ambrosia acanthicarpa
Ragweed, Giant Ambrosia trifida
Ragweed, Short Ambrosia artemisiifolia
Ragweed, Slender Ambrosia confertiflora
Ragweed, Southern Ambrosia bidentata
Ragweed,Western Ambrosia psilostachya
Russian Thistle Salsola kali (pestifer)
Sage, Coastal Artemisia californica
Sage, Pasture Artemisia frigida
Sagebrush, Common Artemisia tridentata
Saltbush, Annual Atriplex wrightii
Shadscale Atriplex confertifolia
Sorrel, Red/Sheep Rumex acetosella
Wingscale Atriplex canescens
Wormwood, Annual Artemisia annua
TREES & SHRUBS:
Acacia Acacia spp.
Alder, European Alnus glutinosa
Alder, Red Alnus rubra
Alder, Tag Alnus incana ssp. Rugosa
Alder, White Alnus rhombifolia
Ash, Arizona Fraxinus velutina
Ash, Green/Red Fraxinus pennsylvanica
Ash, Oregon Fraxinus latifolia
Ash, White Fraxinus americana
Aspen Populus tremuloides
Bayberry Myrica cerifera
Beech, American Fagus grandifolia (americana)
Beefwood/Australian Pine Casuarina equisetifolia
Birch, Black/Sweet Betula lenta
Birch, European White Betula pendula
Birch, Red/River Betula nigra
Birch, Spring Betula occidentalis (fontinalis)
Birch, White Betula populifolia
Box Elder Acer negundo
Cedar, Japanese Cryptomeria japonica
Cedar, Mountain Juniperus ashei (sabinoides)
Cedar, Red Juniperus virginiana
Cedar, Salt Tamarix gallica
Cottonwood, Black Populus balsamifera ssp. trichocarpa
Cottonwood, Eastern Populus deltoides
Cottonwood, Fremont Populus fremontii
Cottonwood, Rio Grande Populus wislizeni
Cottonwood, Western Populus monilifera (sargentii)
Cypress, Arizona Cupressus arizonica
Cypress, Bald Taxodium distichum
Cypress, Italian Cupressus sempervirens
Elm, American Ulmus americana
Elm, Cedar Ulmus crassifolia
Elm, Siberian Ulmus pumila
Eucalyptus Eucalyptus globulus
Hackberry Celtis occidentalis
Hazelnut Corylus americana
Hazelnut, European Corylus avellana
Hickory, Pignut Carya glabra
Hickory, Shagbark Carya ovata
Hickory, Shellbark Carya laciniosa
Hickory, White Carya alba
Juniper, Oneseed Juniperus monosperma
Juniper, Pinchot Juniperus pinchotii
Juniper, Rocky Mountain Juniperus scopulorum
Juniper, Utah Juniperus osteosperma
Juniper, Western Juniperus occidentalis
Locust Blossom, Black Robinia pseudoacacia
Mango Blossom Mangifera indica
Maple, Coast Acer macrophyllum
Maple, Red Acer rubrum
Maple, Silver Acer saccharinum
Maple, Sugar Acer saccharum
Melaleuca Melaleuca quinquenervia (leucadendron)
Mesquite Prosopis glandulosa (juliflora)
Mulberry, Paper Broussonetia papyrifera
Mulberry, Red Morus rubra
Mulberry, White Morus alba
Oak, Arizona/Gambel Quercus gambelii
Oak, Black Quercus velutina
Oak, Bur Quercus macrocarpa
Oak, California Black Quercus kelloggii
Oak, California Live Quercusa agrifolia
Oak, California White/Valley Quercus lobata
Oak, English Quercus robur
Oak, English Quercus robur
Oak, Holly Quercus ilex
Oak, Post Quercus stellata
Oak, Red Quercus rubra
Oak, Scrub Quercus dumosa
Oak, Virginia Live Quercus virginiana
Oak, Water Quercus nigra
Oak, Western White/Garry Quercus garryana
Oak, White Quercus alba
Olive Olea europaea
Olive, Russian Elaeagnus angustifolia
Orange Pollen Citrus sinensis
Palm, Queen Arecastrum romanzoffianum (cocos plumosa)
Pecan Carya illinoensis
Pepper Tree Schinus molle
Pepper Tree/Florida Holly Schinus terebinthifolius
Pine, Loblolly Pinus taeda
Pine, Eastern White Pinus strobus
Pine, Longleaf Pinus palustris
Pine, Ponderosa Pinus ponderosa
Pine, Slash Pinus elliotti
Pine, Virginia Pinus virginiana
Pine, Western White Pinus monticola
Pine, Yellow Pinus echinata
Poplar, Lombardy Populus nigra
Poplar, White Populus alba
Privet Ligustrum vulgare
Sweet Gum Liquidambar styraciflua
Sycamore, Eastern Platanus occidentalis
Sycamore, Oriental Platanus orientalis
Sycamore, Western Platanus racemosa
Sycamore/London Plane Platanus acerifolia
Walnut, Black Juglans nigra
Walnut, California Black Juglans californica
Walnut, English Juglans regia
Willow, Arroyo Salix lasiolepis
Willow, Black Salix nigra
Willow, Pussy Salix discolor

A partial list of some of the common pollen allergy producing trees, grasses and weeds include Acacia, Alder, Ash, Beech, Birch, Cottonwood, Cypress, Elm, Box Elder, Hickory, Juniper, Maple, Mesquite, Mountain Cedar, Mulberry, Olive, Pecan, Red Oak, Sycamore, Walnut, White Oak, Bermuda grass, Brome, Bluegrass, Kentucky Bluegrass, Johnson grass, Meadow Fescue, Mugwort, Orchard grass, Rye, Perrenial Rye, Italian Rye, Red Top, Sweet Vemalgrass, Timothy grass, Wild Oat, Careless Weed, Cocklebur, English Plantain, Lambs Quarter, Marsh Elder, Ragweed, False Ragweed, Giant Ragweed, Short Ragweed, Pigweed, Sagebrush, Tumbleweed, Kochia, Scales, and Yellow Dock.

TABLE 2
Total IgE reactivity to different pollen extracts.

| | | | Pollen |
|---|---|---|---|
| Patient | Cytoplasmic | Commercial | Surface |
| ID # | Extract | extract | Extract |
| 85751 | 5.6 | 5.2 | 3.1 |
| 85843 | 5.9 | 4.3 | 5.1 |
| 85797 | 6.8 | 6.3 | 4.2 |
| 85768 | 7.3 | 4.1 | 3.9 |
| 85752 | 7.3 | 3.3 | 7.5 |
| 85789 | 7.7 | 5.3 | 5.1 |
| 85805 | 9.2 | 7.0 | 5.0 |
| 85794 | 9.3 | 3.8 | 5.6 |
| 85782 | 9.7 | 8.2 | 7.7 |
| 85783 | 9.9 | 6.9 | 2.1 |
| 85792 | 9.9 | 10.3 | 1.3 |
| 85822 | 12.1 | 6.9 | 4.7 |
| 85847 | 15.2 | 6.5 | 4.7 |
| 85760 | 14.3 | 15.2 | 2.7 |
| 85849 | 8.2 | 15.8 | 5.4 |
| 85838 | 16.3 | 12.1 | 3.6 |
| 85785 | 17.8 | 11.7 | 4.3 |
| 85775 | 18.2 | 11.4 | 7.3 |
| 85831 | 22.8 | 15.4 | 5.2 |
| 85841 | 23.0 | 12.7 | 9.9 |
| 85795 | 23.9 | 19.9 | 7.2 |
| 85808 | 31.6 | 28.9 | 7.3 |
| 85799 | 37.9 | 31.8 | 6.9 |
| 85806 | 43.1 | 24.1 | 12.4 |
| Total | 373.1 | 277.1 | 131.9 |

24 individuals, identified in column 1, were screened with the allergen array illustrated in FIGS. 2 and 3 and their total IgE reactivity was calculated by adding IgE reactivity signals for 22 pollen allergens across the indicated extract type (cytoplasmic, pollen surface, or commercial extract).

Cytoplasmic pollen allergen extracts prepared following the methods disclosed herein displayed greater reactivity levels compared to the commercial cytoplasmic extracts as shown in an illustrated example in Table 2 and FIG. 2.

### Example 5

Patient sensitization to pollen allergens. When diagnosing patient sensitization to pollen allergens, arrays with extracted pollen material from multiple plant species disclosed herein offer advantages. For example, when patient responses to a large numbers of allergens are examined using a skin prick test, there is often significant skin irritation and discomfort. In contrast, only a few microliters of patient sera are required to probe a high-density array. Thus, specific and accurate allergic response to multiple pollen allergens can be obtained simultaneously using less sample volume and reducing discomfort to patients.

In examples illustrated in FIG. 2, pollen components extracted from washed and unwashed pollen were dissolved in a buffer containing salts and detergent and spotted in triplicate or duplicate onto SuperEpoxy™ slides by a commercial arraying company (ArrayIt, Inc., Sunnyvale, Calif.). Any solid support capable of affixing proteins, tissues, cells, and lysates is suitable for the construction of the arrays disclosed herein. Three different immunoglobulin standards (IgE, IgG and IgA) were also spotted, as well as human serum albumin, and known recombinant allergens as controls. Following hybridization with human sera, bound antibodies were detected with fluorescently labeled anti-human IgE, IgG, or IgA. The quantity of bound antibody was measured by detecting fluorescence with a high-density detector. Two scanned arrays are illustrated in FIG. 3.

### Example 6

Mapping Pollen Allergy Genes. Diagnostic pollen arrays disclosed herein are used to assess the allergen sensitivity of individual patients. Individuals are categorized according to their pollen sensitization phenotypes including (1) reactivity to any pollen species, (2) reactivity to pollens from the same types of plants, such as grasses (including monocots) or dicots, and (3) reactivity to specific pollen species. Preferably, the entire study population may have more than 100 individuals, even more than 800 individuals, or more than 4000 individuals. As the size of the population increases, the ability to find statistically significant correlations between a particular genetic variant and susceptibility to pollen sensitization also increases.

A sample of DNA of sufficient quantity to perform the desired analysis can be acquired from each individual by any method known in the art. A suitable source and quantity of DNA for this purpose is 10-30 ml blood, since enough DNA can be extracted from leukocytes from such a sample to provide a sufficient quantity of DNA to perform many repetitions of any analysis contemplated herein.

A list of genetic variants is created that will be used to map the pollen allergy phenotypes described above to specific locations in the genome. This list may come from a database of known genetic variants (e.g. dbSNP), may be generated through de novo polymorphism discovery in the study population, or may consist of some combination of the two.

After the list of genetic variants, or “markers”, is established, the variants can be analyzed for their correlation with pollen allergen sensitivity, or diseases related to pollen allergen sensitivity. DNA samples from individuals in the population are analyzed to determine which variants occur in each individual. This analysis can be performed using any method known in the art, including direct sequencing, RFLP methods, allele-specific PCR or SNP genotyping. The resulting catalog of patient genotypes are then correlated to pollen allergy phenotypes using statistical analyses such as linkage mapping or association mapping. In both methods, there is an implicit assumption that the phenotype of interest is either caused by genetic variation or that genetic variations affect the probability that an individual will manifest the phenotype.

Linkage mapping is well known to the skill in the arts and uses related group of individuals. In the simplest form of linkage analysis, two-generation families are collected which contain individuals with the phenotype of interest. At each genetic marker, the inheritance pattern of alleles through the family is compared to the inheritance pattern of the trait. The similarity of the two segregation patterns is quantified by the log odds of linkage or “LOD” score. The statistical properties of the LOD score are well-characterized and rules for declaring significance are well-known to those skilled in the art.

Association studies are conducted with families or with unrelated cases and controls. In brief, a statistical test is used on the distribution of genotypes among patients with a given pollen allergy phenotype (cases), relative to patients without that phenotype (controls), but matched for other variables, such as age, gender, and ethnicity. Where possible, multiple regression analysis can be used to determine interactions among any of the genetic variants. Isolated populations present a suitable case for both linkage and association studies due to the limited allele diversity and pedigree availability.

### Example 7

Identification and isolation of novel allergens. Diagnostic pollen arrays disclosed herein are used to identify and purify specific pollen allergens from mixtures extracted from unwashed or washed pollen. After individual or pooled sera are identified that react with pollen material, that pollen material is further fractionated. Various fractionation procedures are employed, including column chromatography, high pressure liquid chromatography, or electrophoresis. These fractions are spotted into a low or high-density array, and this new array is probed again with sera. Spots that react with the sera are further fractionated to homogeneity, and their components identified through protein sequencing, mass spectrometry, gas chromatography/mass spectrometry, NMR (nuclear magnetic resonance), or other analytical techniques well known to those of skill in the art.

Novel allergens detected in the pollen coat fractions are subjected to a proteomic analysis to identify peptide sequence and used to clone their respective genes. Sufficient material from the pollen coat of the allergenic species is isolated for detection of the relevant proteins on an acrylamide gel stained with Coomassie or other suitable reagents. Western blotting with patient sera is used to confirm the identity of the relevant band. Excision of the band, followed by digestion with specific proteases yields peptides that are sequenced using techniques known to those of skill in the art. After obtaining peptide sequences, GenBank and other relevant databases are searched to identify candidate genes or ESTs. Genes corresponding to completely novel peptides are cloned from cDNA libraries prepared from anthers at a series of developmental stages. Degenerate PCR primers that correspond to the sequenced peptides are used to amplify the pollen coat genes from these cDNAs. Subsequently, 5′ and 3′ RACE experiments are used to characterize the full-length message. A cDNA expression library from anthers at various developmental stages is also constructed to aid in the identification of novel pollen allergens that are reactive to patient sera.

Non-proteinaceous allergens including lipids are identified. Lipids from the pollen coating may serve as irritants that exacerbate the immune response. Lipids extracted from commercially prepared birch and timothy grass pollen induce polymorphonuclear granulocyte migration. The broad role of pollen lipids, particularly those from the pollen coating, has not been generally tested. Lipophilic molecules derived from plants cause contact inflammation and allergy; such molecules include urushiol, a phenolic lipid from poison ivy; and falcarinol, a 17-carbon alkene from English ivy.

Insoluble pollen material is also a source of important allergens. Much of this material is likely to be derived from the sporopollinen that comprises the exine wall, a polymer layer that may contain lipids, carotenoids, and phenolics. Exine walls contain species-specific adhesives. Insoluble material that reacts with sera from several patients is subjected to further analysis to identify the corresponding allergens as disclosed herein. For example, immunoelectron microscopy of pollen sections can ensure that the IgE signal is due to reactivity with exine, rather than from binding to cytoplasmic protein aggregates. Then, the ability of patient sera to cause purified wall fragments to aggregate confirms the presence of allergenic epitopes. Treatment of the exine fragments with different chemical regimes until their ability to bind IgE is destroyed indicates the chemical nature of the epitope. Solubilization of the exine, followed by NMR or mass spectroscopy may be required to identify the allergen. To facilitate this purification and identification process, a closely related ‘control’ pollen that is not recognized by the patient sera may be utilized.

### Example 8

Treatment of allergy including asthma. Diagnostic pollen arrays disclosed herein are useful to identify novel pollen allergens. Pollen is one of the most common triggers of asthma, along with dust mites, mold, and pets. Understanding pollen allergy remains an important health care problem, and understanding its causes represents a promising avenue for the prevention and treatment of asthma.

Most treatments for allergy and asthma require long-term, and often, daily medication to reduce histamine levels, mitigate inflammation and dilate bronchial passages. An improved understanding of the interactions between pollen grains and the cells that line pulmonary epithelia facilitate important advances. For example, the complex mixture of allergens on the pollen surface may directly signal immune cells, triggering previously by uncharacterized responses. New therapeutics may ultimately target such responses. In addition, the lipases that coat the pollen surface may alter the composition of mucosal layers themselves, thereby creating signals that stimulate allergenic response.

Immunotherapy remains a promising alternative in the treatment of pollen allergy. By focusing on compounds that reside on the pollen surface, the compositions and methods disclosed herein can lead to novel drug targets as well as to desensitization immunotherapy to novel groups of pollen allergens. Surveying IgE from the sera of several families, novel correlations that explain the inheritance of a predisposition towards allergy and asthma may be obtained. Immunotherapy with recombinant protein allergens, or with purified epitopes from the pollen wall and coating, may yield treatments with an improved efficacy that are significantly improved relative to the whole-cell pollen extracts in use today.

### Example 9

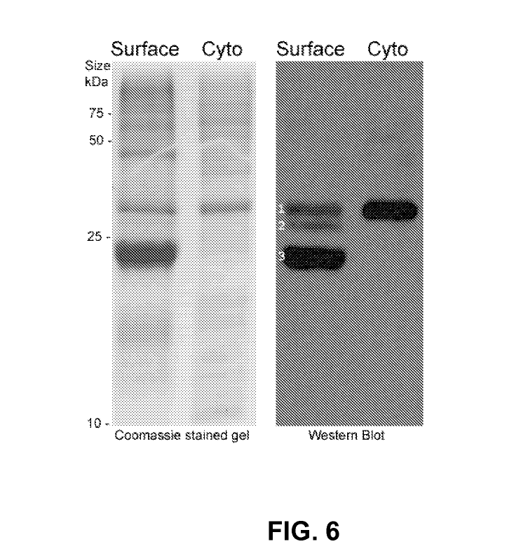

Identification of novel pollen allergens from Bermuda grass pollen through allergen isolation, IgE antibody screening, and sequencing. This example demonstrates that cytoplasmic and surface fractions of pollen extracts obtained using methods disclosed herein can be used to identify specific pollen allergens that cross-react with IgE antibody in sera from individuals. Protein fractions were isolated from the cytoplasmic portions and the surface portion of non-defatted Bermuda grass pollen. Sufficient proteinaceous material from the pollen fractions were analyzed by polyacrylamide gel electrophoresis followed by western blot with pooled sera or stained with Coomassie or other suitable reagents (FIG. 6). Excision of the band, followed by digestion with specific proteases yielded peptides that were sequenced using techniques known to those of skill in the art, e.g., peptide fingerprinting on a MALDI-TOF analyzer. After obtaining peptide sequences, GenBank and other relevant databases were searched to identify candidate genes or ESTs (FIG. 6). The candidate genes include major pollen allergen Cyn d 1, tapetum specific endoxylanase, and cysteine protease from Bermuda grass pollen (FIG. 6). Some of the peptide sequences analyzed are shown in Table 3.

TABLE 3
Peptide sequences of pollen allergens.

| Pollen | | GenBank | Primer sequences to |
|---|---|---|---|
| Allergen | Short Peptide Sequences | Ac. No. | amplify DNA sequence |
| Cysteine | Best Matches: | AY112580 | Forward: |
| protease | YWIVK | (Zea mays) | 1EF0 - |
| (˜22kDa) | SKGAVTPIK | | 5′ CGACTGCGACCCCTACGAC 3′ |
| | PTTVMAWFILVPHCPEK | | 1EF1 - |
| | IRDYVQVPSGEAELQR | | 5′ CGACTGCGACCCCTACGACG 3′ |
| | AVAQQPVAAAVEMGGNLQYYSGGV | | 1EF2 - |
| | -FSGQCGTR (contd.) | | 5′ CGACTGCGACCCCTACGACGG 3′ |
| | | | 1EF3 - |
| | | | 5′ CGACTGCGACCCCTACGACGGC 3′ |
| | Edman sequencing: | | |
| | WVIENGGITTLADYPYR | | |
| | Other Matches | | |
| | KGSTSVK | AK068469 | Reverse: |
| | KQIMWSELS | (Oryza | 1ER0 - |
| | AVWSALSTGEKQQR | sativa) | 5′ TCTGCCCCCACGAGTTCTT 3′ |
| | | | 1ER1 - |
| | | | 5′ TCTGCCCCCACGAGTTCTTG 3′ |
| | VVGGGGAVRGR | CK162 | 1ER2 - |
| | | (Triticum | 5′ TCTGCCCCCACGAGTTCTTGA 3′ |
| | | aestivum) | 1ER3 - |
| | | | 5′ TCTGCCCCCACGAGTTCTTGAC 3′ |

Full length cDNAs or genomic fragments or complete peptide sequences corresponding to the peptide sequences identified herein for the candidate genes shown in FIG. 6 are obtained using techniques known to one of ordinary skill in the art. Degenerate PCR primers that correspond to the sequenced peptides are used to amplify the corresponding genes from a cDNA library or a genomic library. Subsequently, 5′ and 3′ RACE experiments are used to characterize the full-length message. The peptide sequence and the allergenic epitope sequence are identified through any suitable technique known to those of ordinary skill in the art.

Forward primer 5′ CGACTGCGACCCCTACGAC 3′ and reverse primer: 5′ TCTGCCCCCACGAGTTCTTGAC 3′ were used to amplify genomic DNA that correspond to a cysteine protease.

Similarly, allergens can be identified from extracts obtained from other plant species and also from other commercially available extracts. For example, comparison of pollen cytoplasm extracts isolated by the methods disclosed herein with a commercially available pollen extract (Greer Laboratories, Lenoir N.C.) using polyacrylamide gel electrophoresis for plant species showed variations. Polyacrylamide gel electrophoresis (PAGE) of pollen extracts isolated using protocols disclosed herein revealed specific differences with the commercially available extracts, demonstrating the need for improved isolation procedures for pollen components that are disclosed herein, e.g., isolation of pollen components from cytoplasmic and surface fractions of non-defatted pollen.

### Example 10

Hyposensitization or immunotherapy with allergenic pollen components. Hyposensitization or allergy desensitization is an immunotherapy where the patient is desensitized to a particular allergen or a group of allergen by administering progressively higher doses of the allergen of interest. This procedure can either reduce the severity of the allergy response or eliminate hypersensitivity and relies on the progressive skewing of IgG (“the blocking antibody”) production, as opposed to the excessive IgE production seen in hypersensitivity type I cases. It is believed that in allergic reaction the body responds to harmless substances from the environment as if they were invading parasites. The body begins to produce specific immunoglobulin of the E class, IgE. It appears that allergy shots increases the amount of a different class of immunoglobulins, called IgG. It is believed that when IgG molecules circulate in the blood plasma and tissue fluids in large amounts, IgGs bind to allergens and reduce the ability of IgE to detect the presence of the allergens. Thus, the inflammation, secretions, and tissue alterations that take place in untreated allergic disease decrease with immunotherapy. The relative increase of the IgG to IgE ratio results in better tolerance towards the allergen. By giving small but increasing amounts of allergen at regular intervals, tolerance increases and the individual becomes “immune” to the allergens and can tolerate them with reduced symptoms.

Sera from an allergy sufferer or a patient is screened with the diagnostic pollen microarray disclosed herein. An allergy profile of the patient is obtained that quantitatively shows specific allergenic response. Depending on the allergy profile, specific pollen components are identified for hyposensitization or allergy therapy. Small hypodermic syringes are used to inject allergen extracts. Injections are usually given into the loose tissue over the back of the upper arm, half way between the shoulder and elbow. Injections are given under the skin (“subcutaneous”). A suitable range of dosage for hyposensitization experiments or treatments is in the range of about 0.001 microgram to about 1 milligram or 0.01 microgram to 100 microgram. Allergen concentration or dosage depends on the nature of the allergenic response of the patient, which can be evaluated from the allergy profile and also the tolerance levels exhibited by the individual. Allergy injections are started at very low doses. The dose is gradually increased on a regular (and usually weekly) basis, until a “maintenance” dose is reached. This generally translates to four to six months of weekly injections to reach the maintenance dose. After the maintenance dose is reached, the injections are administered less often (every two to four weeks) on a regular basis. Maintenance injections are normally given once per month for a few years.

### Example 11

Personalized treatment plan to pollen allergy. Diagnostic pollen arrays disclosed herein are capable and useful to develop a personalized treatment strategy to treat pollen allergy in individuals. Diagnostic pollen arrays described herein provide an unified platform to test for a wide range of allergenic pollen species and at various concentrations to determine an individual's sensitivity. For example, following screening of an individual's sera with the diagnostic pollen arrays, a subset of pollen allergens are selected using, for example, hierarchical clustering analysis (FIG. 8) either based solely on the antibody reactivity levels and/or on the genetic relatedness of plant species. Pollen extracts or individual pollen allergens including pollen surface allergens and cytoplasmic allergens are pooled or combined to develop a personalized cocktail of pollen allergens to the individual. Varying concentrations of this cocktail are administered at a progressively increasing dose and at varying time periods known to one of ordinary skill in the art. Hyposensitization treatments, as described in Example 12, continue if the patient or physician perceives some benefit. For example, if tolerance levels to administered allergens increase, or if the patient's overall allergy symptoms are diminished or alleviated.

### Materials and Methods

A. Extraction of components from unwashed pollen—pollen coat purification protocol. 10.0 g (or sufficient amount for the specific experiment) of dry pollen (stored at −20° C.) was measured into a 50 mL conical tube. The pollen material was covered with 15 mL (or sufficient amount to resuspend pollen) cyclohexane and vortexed for 5 minutes (typically 5-20 min). The sample was spun in a low speed centrifuge at 3000 rpm at 4° C. for 5 minutes (or other suitable condition to remove particulate material) or filtered. The supernatant was removed and transferred to new tube kept on ice. 15 ml (or sufficient amount to resuspend pollen) cyclohexane was added to the pollen, vortexed for 5 minutes (typically from 5-20 min) and centrifuged for 5 minutes (typically from 5-20 min) or filtered. Both the supernatants were combined for further analysis. The cyclohexane steps were repeated 6 times (typically from 4-8 times) until the supernatant is clear. The remaining pollen material was preserved for extracting the cytoplasmic fraction. The combined supernatant was spun or filtered to remove any remaining pollen and the supernatant was transferred to a fresh tube.

The cyclohexane was evaporated by passing air over it, in the fume hood until about 1 ml of cycleohexane is left. About 1 ml of the remaining cyclohexane was transferred into a 2.0 ml Eppendorf™ tubes (or any suitable centrifuge tubes) and was evaporated until about 0.75 ml cyclohexane remains. (Alternatively, the cyclohexane can be evaporated completely and the pollen coat residue can be resuspended in a detergent-containing buffer, such as TBS-T). An equal volume of 20% TCA (trichloroacetic acid) or 80% ice-cold acetone was added to the sample. The sample was vortexed for 30 minutes (typically from 14-60 min) at 4° C. and spun in a centrifuge at 14,000 rpm (can vary from 10,000-14,000 rpm) at 4° C. for 15 minutes (typically from 15-30 min). All the samples were maintained on ice. About 300 μL cold acetone (or a suitable amount to achieve protein precipitation) was added to the aqueous layer. Optionally, the sample is vortexed briefly and then incubated at −20° C. for at least 1 hour. The sample is spun for 5 minutes at 4° C. (or other suitable condition to collect the precipitate). The supernatant is removed, the pellet is washed with cold 80% acetone until white, and dried. The pellet was resuspended in a suitable buffer and the pH of the final sample was adjusted to about pH 7.0. Optionally, the pellet is resuspended in 40 μl SDS PAGE loading buffer and titrated with 1.0M NaOH in 1 μl increments until blue color returns. Resuspension buffer may vary depending on what the intended us of the extracts. The pellets can, for example, be resuspended in TBS-T or PBS-T.

B. Extraction and purification of cytoplasmic components from washed pollen. Cyclohexane-washed pollen pellet was suspended in TBS-T (20 mM Tris, 136 mM NaCl, 0.1% Tween 20, pH 7.5). If desirable, the suspended pellets can be frozen in liquid nitrogen and thawed immediately; this step is typically repeated twice. The sample was then transferred to a mortar and was ground with a pestle (or other suitable device that will cause the pollen to break) consistently for 7 minutes (or until the majority of the pollen is lysed). The residue was transferred to microfuge tubes (or other tubes suitable for centrifugation). The tubes were spun at 14,000 rpm (typically from 10,000-14,000 rmp) for 5 minutes (typically 5-15 minutes) in a centrifuge. The supernatant was transferred to another tube and 1:100 protease inhibitors were added and, if desired, the samples were stored at 4° C. Equal volumes of 20% TCA or 80% acetone were added to the samples. The samples were incubated for about 30 minutes (typically 15-120 min) on ice and were spun for 15 minutes (typically 10-30 min) at 4° C. The supernatant was removed and the pellet was washed with 80-100% acetone. The washed pellet was incubated on ice for 10 minutes was then spun for 5 minutes at 4° C. The supernatant was removed and the pellet was stored at 4° C. until the next step. The pellet was resuspended in a suitable buffer and the pH of the final sample was adjusted to about pH 7.0.

C. Preparation of low-density arrays. Proteins were quantified using a suitable assay kit (such as Coomassie Plus™—The Better Bradford Assay Kit, catalog #23236, Pierce, Rockford, Ill.). About 2 μg (typically 0.5-5 μg) of pollen components were spotted in duplicates onto nitrocellulose membrane. Also about spot 2 μl (typically 0.5-5 μg) of undiluted serum and 1 μl (typically 0.5-5 μg) of undiluted 2° Ab were spotted in duplicates as positive controls. The filter was washed with TBS-T (20 mM Tris, 136 mM NaCl, 0.1% Tween 20, pH 7.5) for 15 minutes (typically 10-60 min). The filter was incubated at about 20° C. for 1 hour (typically 30-180 min) in blocking buffer (5% nonfat dried milk or 1% bovine serum albumin in TBS-T). The filter was incubated overnight at 4° C. in the same buffer along with the addition of antisera (diluted 1:2 to 1:100, as necessary). After incubation, the filter was washed with TBS-T, 3 times for 5 minutes each (or a suitable number of times to rid filter of unbound proteins). A secondary antibody, such as goat anti-human IgE conjugated to HRP (Catalog #48-139-H, Antibodies Incorporated, Davis, Calif.) was added at a suitable dilution (1:100 to 1:1000) and was incubated for about 1 hour (typically 60-180 min) at room temperature in TBS-T or blocking buffer. The membrane was washed with TBS-T, 3 times for 15 minutes each (or a suitable number of times to rid filter of unbound proteins). Bound antibodies were detected using an ECL kit (Amersham Life Science, catalog #1059243 and #1059250) according to the manufacturer's instructions.

D. High-density array. Proteins were quantified using a suitable assay kit (such as Coomassie Plus™—The Better Bradford Assay Kit, catalog #23236, Pierce, Rockford, Ill.). About 0.5-1.5 nl of pollen components dissolved at 0.3-1 μg protein/μl into Protein Printing Buffer (#PPB, ArrayIt, Sunnydale, Calif.) were spotted in triplicates using NanoPrint™ Microarray System onto SuperEpoxy™ slides with about 0.5-1.5 nL drop size. Serial dilutions (1 pg-800 pg) of purified IgE (IgE Myeloma Serum, catalog # 30-A105, Fitzgerald Industries International, Concord, Mass.) IgG, (Intact Human IgG, catalog #P80-105, Bethyl Laboratories Inc., Montgomery, Tex.), and IgA (Human IgA, catalog #P80-102, Bethyl Laboratories Inc., Montgomery, Tex.) as well as 0.5-1.5 nL of Human Serum Albumin (catalog# 05420-500MG, Sigma, St. Louis, Mo.), and Bet v 1, Phl p 2, Amb a 1, Alt a 1, and Der p 1 (Biomay, Vienna, Austria) dissolved at 0.3-1.0 μg/μL were spotted in triplicates. Slides were washed with 1×PBS-T or TBS-T on SpeciMix™ (or suitable mixer) for 10 minutes 3 times (reagent: 3×5 mL PBS-T or TBS-T). Non-specific binding was limited by treating slides with BlockIt™ buffer (or any other suitable blocking buffer) on SpeciMix™ (or suitable mixer) for 120 minutes (reagent: 3 mL BlockIt). Slides were rinsed with 1×PBS-T or TBS-Ton SpeciMix™ (or suitable mixer) for 5 minutes 5 times (reagent: 5×5 mL PBS-T or TBS-T). Slides were incubated with human sera in ProPlate using 1:1 dilutions (can vary from undiluted to 1:100) for 60 minutes @ room temperature, overnight @ 4° C. Sera should be diluted in PBS-T or TBS-T containing 1% HSA (reagent: 48 μL PBS-T with 1% HSA+sera/well). Be sure not to cross-contaminate from well to well. Sera was aspirated and PBS-T or TBS-T was added to each well 3× as an initial wash (reagent: 3×100 μL PBS-T or TBS-T per well). Slides were removed from ProPlate and washed with 1×PBS-T or TBS-T SpeciMix™ (or suitable mixer) for 10 minutes 3 times (reagent: 3×5 mL PBS-T). Slides were incubated with 20 Ab on SpeciMix™ (or suitable mixer) using 1:50-1:1000 (IgE) and 1:100-1:100,000 (IgG) dilutions. Antibody should be diluted in PBS-T containing 1% HSA for 120 minutes (reagent: 2 mL PBS-T or TBS-T with 1% HSA+2° Ab). Slides were washed with PBS-T or TBS-T on SpeciMix™ (or suitable mixer) for 10 minutes two times (reagent: 2×5 mL PBS-T) and with PBS or TBS on SpeciMix™ (or suitable mixer) for 10 minutes twice (reagent: 2×5 mL PBS). Slides were rinsed with ddH₂O for a few seconds and dried by spinning in low speed centrifuge and stored in the dark until scanning. Slides are scanned with GenePix™ 4000B and images are converted into data using GenePix™ Pro 6.0.1 software.

E. Mapping pollen allergy genes. Sera from individual patients are hybridized to pollen arrays, and bound antibodies are detected with anti-IgE, anti-IgG, or anti-IgA, as described herein. Using a microarray scanner, fluorescent signals corresponding to each spot on the hybridized array are calculated. For each signal above a background threshold, a quantitative score to indicate patient sensitivity is assigned. Each score, or groups of scores, constitutes a sensitivity phenotype.

For individuals surveyed, genotype data (SNPs, microsatellies, indels) are collected corresponding to informative markers distributed across the genome. The LOD-based linkage analysis of families is used to identify regions in the genome that correlate with sensitivity phenotypes. In some cases, more specific mapping methods, including homozygosity by descent mapping, can be used. When useful, association mapping methods are employed to narrow regions of interest and identify alleles that correlate with the phenotype of interest.

F. Protocol for Method of Identifying Novel (Pollen) Allergens: One of the first steps is to isolate proteins from desired tissues, e.g., from pollen cytoplasm or pollen surface. Isolated and purified protein fractions are electrophoresed on a separating gel (Poly Acrylamide Gel Electrophoresis), preferably in duplicates. Then, one of the gels is stained with a visualizing agent, for example, Coomassie, Amido Black, Sypro Red, or any suitable visualizing or labeling agent or dye. The electrophoresed fractions in the second gel are transferred on to a blotting membrane as in a standard western blotting procedure disclosed herein. The membrane is then probed with pooled human sera as the primary antibody in 1:1 antibody:blocking agent mixture. The primary antibody-bound membrane is then hybridized with anti-human IgE conjugated to a visualizing agent (calorimetric, chemiflourescent, or chemiluminescent), in a ratio appropriate to the blot size (e.g., 1:200 antibody:blocking agent is suitable). The hybridization reaction is visualized after developing an exposed film to detect the fluorescent or radioactive or chemiluminescent reactions. The reactive bands on the blot are matched to the corresponding ones on the gel. The bands from the gel are cut out and sequences are analyzed from the purified peptide product. The resulting sequences are compared against other proteins in the database to obtain a preliminary understanding of their structure and function.

G. Western Blot Protocol: Gel to Membrane Blotting: 1. Whatman™ filter papers and one Immobilon-P (PVDF) membrane were cut according to the size of the gel. Immobilon-P membrane was soaked in 100% methanol for 15 seconds and then was transferred to ddH₂O for 2 min followed by equilibration in semi-dry transfer buffer (sdTB) for 5 min. The gel was removed from plate sandwich noting the appropriate left to right orientation. The gel was rinsed briefly in a semi-dry transfer buffer (sdTB). sdTB-wetted filter papers were layered as a platform of blotter. The immobilon-P membrane was positioned on top noting the orientation to match the gel orientation. The gel was placed in known orientation onto Immobilon-P membrane. More wetted filters papers were placed on top and the air bubbles were removed by rolling a smooth surfaced object. The top of the electro-blotter was installed and the system was run at 150 mA for 45 min. The filter papers were removed and the sizes of the standards were checked to confirm blotting. The gel was removed and slits in the membrane were cut at 75, 50, 25, and 10 kDa.

(i) Probing the Membrane: The transferred membrane was rinsed in TBSt (0.05% Tween 20 in TBS) for about 5 min. The membrane was blocked 10-20 ml of blocking solution for at least one hour at room temp with gentle agitation. Blocking Solution: 3% BSA in TBSt buffer. The blocking solution was decanted and the membrane was washed with TBSt for 5 min with gentle agitation. The wash solution was decanted and the tubes were incubated with primary antibody (diluted in blocking Solution 1:1) overnight at 4° C. with gentle agitation. Primary antibody solution was decanted and the membrane was washed in TBSt for 5 min. The washes were repeated twice for a total wash time of 15 min. The wash solution was decanted and conjugated secondary antibody was added that was, diluted in blocking solution 1:20. The system was incubated for 1 hr with gentle agitation. The conjugate solution was decanted and the system was washed in TBSt for 5 min. This step was repeated and a final 5 min-wash was performed in TBS to remove residual detergent.

(ii). Developing the Membrane: Equal parts of ECL (chemiluminescent) solutions A and B (about 1 ml each per membrane) were added. ECL solution mixture was spread onto protein side of membrane and was incubated for 5 min The filter was drained and wrapped in a plastic wrap and was exposed to X-ray film. Exposure times ranged from 30 sec to 10 min. The exposed films were developed in a developer as required by the manufacturer.

Primary antibody used was a pooled sera from 500 people and the secondary antibody used was a mouse anti-human IgE coupled to HRP (horse radish peroxidase).

Semi-dry Transfer Buffer (Bjerrum and Schafer-Nielsen)

| Concentration | For 1 liter add |
|---|---|
| 48 mM Tris | 5.82 g |
| 39 mM glycine | 2.93 g |
| 0.0375% SDS or | 0.375 g or 3.75 ml of 10% SDS |
| 20% MeOH | 200 ml |
| ddH₂O | Add to 1 liter final volume |

H. Nucleic Acid Analysis

(i) Pollen DNA Extraction

About 20-30 μl of pollen was ground for 3 min. in Shorty Buffer. About 0.55 ml shorty buffer was added and 0.55 ml phenol chloroform mix was added to the sample. The sample was vortexed for 20 seconds and was transferred to ice. The sample was spun for 5 min at 14000 rpm at room temperature. The supernatant was transferred to a tube with 0.55 ml phenol chloroform mix and vortexed for 10 seconds and then were transferred to ice. The supernatant was spun for 5 min at room temperature. The supernatant was transferred to a tube with 0.5 ml isopropyl alcohol and was mixed by inversion. The sample was allowed to precipitate at room temperature and was then spun for 10 min. The pellet was rinsed with 70% EtOH and then dried for 30 min at room temperature. The pellet was resuspended in 100 μl Tris-EDTA (TE) and the tube was transferred to a shaker at 200 rpm at 37° C. for one hour.

Shorty Buffer (500 mL) includes glycogen (10 mg/L)-500 L; 1M Tris-HCl, pH 9.0-100 mL; 2M LiCl-100 mL; 0.5M EDTA-25 mL; 10% SDS-50 mL; and H₂O-225 mL. (P. J. Krysan et al., (1999).

(ii) PCR Amplification of Pollen DNA

PCR primers for each identified allergen were designed using highly conserved portions of cDNA sequences between corn and rice. The PCR reaction conditions included the following parameters:
1. 95° for 30 s (92-98°, 10 s-3 min)
2. 92° for 15 s (92-98°)
3. 65° for 15 s (50-70°)
4. 72° for 30 s (65-72°; 10 s-3 min)
5. Go to #2 for 9 times (5-30 times)
6. 92° for 15 s (92-98°)
7. 67° for 15 s (50-70°)
8. 72° for 30 s (65-72°)
9. Go to #6 for 29 times (5-30 times)
10. 72° for 10 min (65-72°)
11. End

(ii) Cloning and Sequencing:

The PCR products were run on a 1% agarose gel. The DNA bands of the appropriate size were cut out and cleaned using a Qia-Quick™ gel extraction kit from Qiagen™. DNA was cloned into a TOPO TA Cloning Vector™ from Invitrogen according to the kit instructions. Transformed colonies were sequenced via standard methods by in a nucleic acid sequencing facility.

### DOCUMENTS

These documents are incorporated by reference to the extent they relate materials or methods cited in the present application.
Abney et al., Quantitative-trait homozygosity and association mapping and empirical genomewide significance in large, complex pedigrees: fasting serum-insulin level in the Hutterites. Am J Hum Genet, 2002. 70(4): p. 920-34.
Bollag et al., Protein Methods. 1996, New York: Wiley-Liss. 415.
Dennison, C., A guide to protein isolation. Focus on Structural Biology, 2003,
Boston: Kluwer Academic Publishers. 248.
Krysan et al., T-DNA as an Insertional Mutagen in Arabidopsis. The Plant Cell, vol. II: (1999) 2283-2290.
Mayfield et al., Gene families from the Arabidopsis thaliana pollen coat proteome. Science, 2001. 292 (5526): p. 2482-5.
Ogren, T. L., The Revolutionary Guide to Healthy Landscaping, Ten Speed Press, (Jun. 1, 2000).
Strachan, T. and A. Read, Human Molecular Genetics 2. 2nd ed. 1999, New York: Wiley-Liss. 576.

We claim:

1. A pollen array comprising a pollen surface allergen.

2. The pollen array of claim 1, wherein the array is a diagnostic pollen array.

3. The pollen array of claim 1, wherein the allergen is reactive to an IgE antibody.

4. The pollen array of claim 1, wherein the allergen is present in a pollen extract.

5. The pollen array of claim 4, wherein the pollen extract comprises a pollen cell surface extract.

6. The pollen array of claim 4, wherein the pollen extract comprises a pollen cytoplasmic extract

7. The pollen array of claim 5, wherein the cell surface extract is obtained using an organic solvent.

8. The pollen array of claim 6, wherein the pollen cytoplasmic extract is obtained using an aqueous solvent.

9. The pollen array of claim 1, wherein the allergen is a recombinant pollen allergen.

10. The pollen array of claim 1, wherein the allergen is substantially pure.

11. The pollen array of claim 1, wherein the allergen is highly pure.

12. The pollen array of claim 1, wherein the allergen is a peptide.

13. The pollen array of claim 1, wherein the allergen is a multimeric pollen allergen.

14. The pollen array of claim 1, wherein the allergen is present in a concentration of about 0.05-1.0 μg/μL.

15. The pollen array of claim 1, wherein the allergen is present in an amount sufficient to detect an allergy response in a patient sample.

16. The pollen array of claim 1 comprising a plurality of pollen allergens spotted at a density of about 100 spots per square inch to about 100,000 spots per square inch.

17. The pollen array of claim 1 comprising a plurality of pollen allergens spotted at a density of about 1000 spots per square inch to about 10,000 spots per square inch.

18. The pollen array of claim 1 further comprising a pollen allergen selected from the group consisting of cytoplasmic, cell-wall bound, and membrane bound allergens.

19. The pollen array of claim 1, wherein the allergen is selected from the group consisting of allergenic plant species Wal (Walnut), Ber (Bermuda grass), Scy (Sycamore), Orc (Orchard grass), Pec (Pecan), Tim (Timothy grass), Olv (Olive), Mug (Mugwort), WOk (White Oak), Rag (Ragweed), Ald (Alder), Eld (Box Elder), Cot (Cottonwood), Mul (Mulberry), Jhn (Johnson grass), Elm, Ash, Ced (Cedar), Blu (Bluegrass), Bir (Birch), Rye, and ROk (Red Oak).

20. The pollen array of claim 1 comprising at least one cytoplasmic allergen and an antibody standard.

21. A method of making a pollen array, the method comprising:
(a) obtaining pollen from an allergenic plant species;
(b) preparing a pollen cell surface extract from the pollen;
(c) obtaining at least one pollen allergen from the pollen extract; and
(d) applying the pollen allergen to a solid support in an orderly fashion in the array.

22. The method of claim 21, wherein the pollen is untreated and unwashed.

23. The method of claim 21, wherein the pollen is non-defatted.

24. The method of claim 21, wherein the pollen cell surface extract is prepared using an organic solvent selected from the group consisting of cyclohexane, hexane, diethylether, formamide, dimethylformamide, dimethyl sulfoxide, acetone, ethyleneglycol monomethyl ether, toluene, benzene, hydrocarbon solvents and halogenated solvents.

25. The method of claim 21, wherein the solid support is selected from the group consisting of glass, epoxy-coated glass, plastic, nylon and nitrocellulose membrane.

26. A method of preparing a pollen surface allergen extract, the method comprising:
(a) obtaining an unwashed or untreated pollen from an allergenic plant species;
(b) exposing the unwashed or untreated pollen to an organic solvent to separate the surface pollen; and
(c) obtaining the surface pollen allergen extract.

27. A pollen surface extract prepared by the method of claim 26.

28. The pollen extract of claim 27 is substantially free of cytoplasmic components.

29. The pollen extract of claim 27 is substantially completely free of cytoplasmic components.

30. A method of preparing a pollen extract comprising pollen surface allergens and pollen cytoplasmic allergens, the method comprising:
(a) obtaining pollen from an allergenic plant species;
(b) washing the pollen with an organic solvent to separate the surface pollen allergens;
(c) lysing the washed pollen in an aqueous solution to obtain the cytoplasmic allergens;
(d) obtaining the pollen extract comprising the pollen surface allergens and pollen cytoplasmic allergens.

31. A method to measure antibody levels to pollen allergens in an individual, the method comprising:
(a) providing the pollen array of claim 1 comprising a pollen surface allergen;
(b) applying a serum sample from the individual to the pollen array; and
(c) measuring antibody levels to the allergen by quantifying allergen-antibody reactions on the array.

32. A method of measuring sensitivity to pollen allergens in an individual, the method comprising:
(a) providing the pollen array of claim 1 comprising at least one pollen surface allergen;
(b) applying a serum sample from the individual to the pollen array; and
(c) analyzing the sensitivity to pollen allergen by measuring antibody levels to the allergen.

33. A method of identifying a genetic locus that contributes to allergy, the method comprising:
(a) diagnosing allergen sensitization in a group of individuals using the pollen array of claim 1;
(b) correlating results of the diagnosing to at least one genetic marker linked to a locus; and
(c) identifying the genetic locus that contributes to allergy.

34. A method to develop a pollen-specific allergy treatment in an individual, the method comprising:
(a) providing the pollen array of claim 1 comprising at least one pollen surface allergen;
(b) identifying a pollen allergen or a group of pollen allergens that contribute to allergy in the individual; and
(c) developing a therapy to treat allergy to the pollen allergen in the individual.

35. The method of claim 34, wherein the group of pollen allergens are selected based on their reactivity to the individuals IgE antibodies.

36. The method of claim 34, wherein the treatment is personalized to the individual.

37. A method to treat an individual to one or more specific pollen allergens, the method comprising:
(a) providing the pollen array of claim 1 comprising at least one pollen allergen to develop an allergy profile of the individual;
(b) administering one or more pollen allergens identified in (a) at a dosage that is sufficient to elicit a desensitization response in the individual; and
(c) treating the individual by administering progressively higher doses of allergen and monitoring the hyposensitization response in the individual to the pollen allergen using the pollen array.

38. An isolated allergenic pollen peptide from Bermuda grass comprising an amino acid sequence WVIENGGITTLADYPYR.

39. The allergenic pollen peptide of claim 38 is synthetic.

40. The allergenic pollen peptide of claim 38 is substantially free of other allergenic peptides.

41. An immunologically active composition comprising the allergenic peptide of claim 38.

42. The immunologically active composition of claim 41 comprises the allergenic peptide in an amount that is effective to induce hyposensitization in an individual.

43. The allergenic pollen peptide of claim 38 comprising an amino acid sequence that is more than 95% identical.

44. The allergenic pollen peptide of claim 38 comprising an amino acid sequence that is more than 90% identical.

45. The allergenic pollen peptide of claim 38 comprising an amino acid sequence that is more than 85% identical.
